(19) United States
(12) Patent Application Publication
(10) Pub. No.: US 2022/0288022 A1
(43) Pub. Date: Sep. 15, 2022

(54) PHARMACEUTICAL COMPOSITION FOR TOPICAL USE THAT IS IN THE FORM OF A DISPERSED PHASE BASED ON AT LEAST ONE SHORT DIOL IN A CONTINUOUS FATTY PHASE AND COMPRISING AT LEAST ONE ANTI-INFLAMMATORY SUBSTANCE

(71) Applicant: SOCIETE D'EXPLOITATION DE PRODUITS POUR LES INDUSTRIES CHIMIQUES SEPPIC, Paris Cedex 07 (FR)

(72) Inventors: Catherine BULCOURT, LA GARENNE COLOMBES (FR); Alice DENIS, CASTRES (FR); Séverine SIGURANI, CASTRES (FR)

(21) Appl. No.: 17/632,664

(22) PCT Filed: Aug. 4, 2020
(86) PCT No.: PCT/FR2020/051430

§ 371 Date: Feb. 3, 2022

(30) Foreign Application Priority Data
Aug. 9, 2019 (FR) FR1909117

(51) Int. Cl. A61K 31/404; A61K 9/00; A61K 9/107; A61K 47/10; A61K 31/196; A61K 31/216; A61K 31/381; A61K 31/192; A61K 47/34

(52) U.S. Cl. CPC A61K 31/404; A61K 9/0014; A61K 9/107; A61K 47/10; A61K 31/196; A61K 31/216; A61K 31/381; A61K 31/192; A61K 47/34

(57) ABSTRACT

Topical pharmaceutical composition as a dispersed phase based on a short diol in a continuous fatty phase and including an anti-inflammatory substance, the composition including: a gelled phase including an anti-inflammatory substance and a diol including 3-8 carbon atoms and represented either by formula (Iₐ): R^{a}₁—(R^{b}₁)(OH)—(OH)(R^{c}₁)(R^{d}₁) (Iₐ), in which each of the radicals R^{a}₁, R^{b}₁, R^{c}₁ and R^{d}₁ represents, independently of each other, a hydrogen atom or a saturated aliphatic radical including 1-5 carbon atoms, or by formula (I_{b}): R^{a}₁—C(R^{b}₁)(OH)—[C(R^{e}₁)(Rf₁)]ₜ—C(OH)(R^{c}₁)(R^{d}₁) (I_{b}), t=1, 2 or 3, each of the radicals R^{a}₁, R^{b}₁, R^{c}₁ R^{d}₁, R^{e}₁ and R^{f}₁ represents, independently of each other, a hydrogen atom or a saturated aliphatic radical including 1-5 carbon atoms. The radicals R^{a}₁or R^{b}₁ and/or the radicals R^{c}₁or R^{d}₁does not represent a hydrogen atom, a fatty phase including an oil and an emulsifying system including a combination of emulsifying surfactants.

### BACKGROUND OF THE INVENTION

### Field of the Invention

The present invention relates to a pharmaceutical composition for topical use which is in the form of a dispersion of a gelled phase including at least one diol consisting of 3 to 8 carbon atoms and comprising at least one anti-inflammatory substance in an oily phase, and intended to be used in human or animal therapy.

### Description of the Related Art

Dermopharmaceutical and pharmaceutical compositions may be in the form of aqueous solutions, emulsions and powders.

An emulsion is an initially heterogeneous mixture of two mutually immiscible liquid substances, the two substances being called “phases”. Said mixture becomes macroscopically homogeneous via an external operation, for instance mechanical stirring with a suitable speed and spindle, or the addition of an amphiphilic substance called a surfactant. When presented in macroscopically homogeneous form, the mixture consists of a discontinuous phase, dispersed in the other phase which is called the “continuous phase”.

Emulsions are preferred forms since they make it possible to convey both the water-soluble substances and the liposoluble substances that are commonly used in these applications. A distinction is made between oil-in-water (O/W) emulsions in which the continuous phase consists of a hydrophilic phase, generally an aqueous phase, and the dispersed phase consists of a lipophilic fatty phase, and water-in-oil (W/O) emulsions in which the continuous phase consists of a lipophilic fatty phase and the dispersed phase consists of a hydrophilic phase, generally an aqueous phase.

Oil-in-water emulsions are intrinsically more stable than water-in-oil emulsions; water-in-oil emulsions nevertheless have a number of advantages. Specifically, the separation between the water droplets reduces the possibility of the proliferation of microorganisms. Furthermore, the use of preserving agents, which is essential when the continuous phase is aqueous, can be avoided or reduced when the continuous phase is fatty. Water-in-oil emulsions are far less sensitive to low temperature than oil-in-water emulsions. Lastly, for topical applications for cosmetic use, the European patent application published under the number EP 1961455 A1 discloses that an oily continuous phase makes it possible to cover the skin after application of the water-in-oil emulsion, which protects the skin from dehydration and against external substances by forming a persistent oily film, thus making it possible to treat dry skin.

The solutions proposed in the prior art for preparing dermatological emulsions which are in the water-in-oil form are not satisfactory either because the silicone derivatives employed are volatile and may have harmful effects with regard to the environment and the users, or because the silicone derivatives employed are sparingly volatile and they then give unpleasant sensory properties after topical application, for instance tacky sensations on the skin. Furthermore, when they comprise particular therapeutic substances, for instance particular nonsteroidal anti-inflammatory substances (or NSAIDs) and more particularly arylacetic (or arylalkanoic) derivatives and 2-arylpropionic acids (or profens), these emulsions of water-in-oil type display problems of stability on storage or even an inability to achieve a stable water-in-oil form.

On this basis, one problem is to develop a novel composition which is in the form of an emulsion of water-in-oil type, which does not feature the drawbacks exposed above, which remains homogeneous at room temperature (25° C.) and at 45° C. after storage for a minimum of three months, and which comprises, as active therapeutic agent, substances chosen from elements of the group consisting of specific nonsteroidal anti-inflammatory agents (or NSAIDs) and more particularly of arylacetic (or arylalkanoic) derivatives and 2-arylpropionic acids (or profens).

### SUMMARY OF THE INVENTION

This is why the inventors have sought to develop novel dispersions in which the continuous phase is oily and the dispersed phase is polar but nonaqueous, free of added water, which do not have the drawbacks described above, and which remain homogeneous at room temperature (greater than or equal to 20° C. and less than or equal to 25° C.) and at 45° C. after storage for at least three months.

A solution of the present invention is a pharmaceutical composition (E1) for topical use comprising a gelled phase (A1) free of added water and dispersed in a continuous phase (A2), said pharmaceutical composition (E1) comprising:
a gelled phase (A1) comprising at least one anti-inflammatory substance (AI) and at least one diol including from 3 to 8 carbon atoms and represented either by formula (Iₐ):

R^{a}₁—C(R^{b}₁)(OH)—C(OH)(R^{c}₁)(R^{d}₁) (Iₐ),

in which each of the radicals R^{a}₁, R^{b}₁, R^{c}₁ and R^{d}₁ represents, independently of each other, a hydrogen atom or a saturated aliphatic radical including from 1 to 5 carbon atoms, or by formula (I_{b}):

R^{a}₁—C(R^{b}₁)(OH)—[C(R³₁)(R^{f}₁)]ₜ—C(OH)(R^{c}₁)(R^{d}₁) (I_{b}),

in which t is equal to 1, 2 or 3, each of the radicals R^{a}₁, R^{b}₁, R^{c}₁, R^{d}₁, R^{e}₁ and R^{f}₁ represents, independently of each other, a hydrogen atom or a saturated aliphatic radical including from 1 to 5 carbon atoms, it being understood that at least one of the radicals R^{a}₁ or R^{b}₁ and/or at least one of the radicals R^{c}₁ or R^{d}₁ do not represent a hydrogen atom;
a fatty phase (A2) comprising at least one oil and an emulsifying system (S) comprising a combination of at least one emulsifying surfactant (S1) and at least one emulsifying surfactant (S2).

Preferably, the pharmaceutical composition (E1) for topical use will comprise, per 100% of its mass:
from 60% to 98% by mass, more particularly from 60% to 95% by mass, and even more particularly from 60% to 90% by mass, of the gelled phase (A1), and
from 2% to 40% by mass, more particularly from 5% to 40% by mass, and even more particularly from 10% to 40% by mass, of the fatty phase (A2).

The emulsifying surfactant (S1) is preferably selected from the elements of the group consisting of alkylpolyglycoside compositions and compositions of alkylpolyglycosides and of fatty alcohols, and the emulsifying surfactant (S2) is preferably selected from the elements of the group consisting of polyglycerol esters, alkoxylated polyglycerol esters, polyglycol polyhydroxystearates, polyglycerol polyhydroxystearates and alkoxylated polyglycerol polyhydroxystearates.

For the purposes of the present invention, the term “anti-inflammatory substance (AI)” means a chemical substance which is used for combating inflammation, the body's process of defence against external attack, characterized by the appearance of signs of heat, pain, redness and tumefaction.

Among the anti-inflammatory substances (AI), steroidal anti-inflammatory substances and non-steroidal anti-inflammatory substances are distinguished.

According to a particular aspect, the anti-inflammatory substances (AI) of the present invention are non-steroidal anti-inflammatory substances, more particularly arylacetic (or arylalkanoic) derivatives and 2-arylpropionic acids (or profens).

The term “for topical use” used in the definition of the composition (E1) according to the invention, which is in the form of a dispersion of a gelled phase (A1) in a continuous phase (A2) and as defined above, means that said composition (E1) is employed by application to the skin, the hair, the scalp or mucous membranes, whether it is a direct application in the case of a cosmetic, dermocosmetic, dermopharmaceutical or pharmaceutical composition or an indirect application, for example in the case of a body hygiene product in the form of a textile or paper wipe or of health products intended to be in contact with the skin or mucous membranes.

For the purposes of the present invention, the term “gelled phase (A1)” denotes a homogeneous phase characterized in that the dynamic viscosity of said phase (A1), measured at a temperature of 20° C. and using a Brookfield LVT viscometer at a speed of 6 rpm, is greater than or equal to 1000 mPa·s and less than or equal to 100 000 mPa·s, more particularly greater than or equal to 10 000 mPa·s and less than or equal to 100 000 mPa·s. Depending on the case, the composition (E1) according to the invention may have one or more of the following features:
said composition (E1) comprises, per 100% of its mass:
   from 0.625% to 10% by mass, more particularly from 0.625% to 8.5% by mass and even more particularly from 0.625% to 7% by mass of a crosslinked anionic polyelectrolyte (AP1),
   from 0.625% to 5% by mass, more particularly from 0.625% to 3.5% by mass, and even more particularly from 0.625% to 2% by mass, of at least one anti-inflammatory substance (AI), and
   from 85% to 98.75% by mass, more particularly from 88% to 98.75% by mass and even more particularly from 91% to 98.75% by mass of at least one diol including from 3 to 8 carbon atoms and represented either by formula (Iₐ) as defined previously, or by formula (I_{b}) as defined previously.

In the definition of the composition according to the invention, it should be noted that the term “crosslinked anionic polyelectrolyte (AP1)” denotes a nonlinear crosslinked anionic polyelectrolyte, which is in the form of a three-dimensional network which is insoluble in water but swellable in water and which leads to the production of a chemical gel.
the diol including from 3 to 8 carbon atoms and represented either by formula (Ia) or by formula (Ib) is chosen from 1,2-propanediol, 1,2-butanediol, 1,3-butanediol, 1,2-pentanediol, 1,2-hexanediol, 1,2-octanediol, 2,3-butanediol, 2,3-pentanediol, 2,3-hexanediol, 2,5-hexanediol or 2-methyl-2,4-pentanediol.
the diol including from 3 to 8 carbon atoms and represented either by formula (Ia) or by formula (Ib) is chosen from 1,2-propanediol, 1,2-butanediol, 1,3-butanediol, 1,2-pentanediol, 1,2-hexanediol or 2-methyl-2,4-pentanediol.
the crosslinked anionic polyelectrolyte (AP1) comprises a proportion of greater than or equal to 25 mol % of monomer units derived from 2-methyl-2-[(1-oxo-2-propenyl)amino]-1-propanesulfonic acid in free acid or partially or totally salified form.
the crosslinked anionic polyelectrolyte (AP1) comprises:
   (a2) optionally, a proportion of greater than 0 mol % and of less than or equal to 75 mol % of monomer units resulting from at least one monomer chosen from the elements of the group consisting of acrylamide, N,N-dimethylacrylamide, methacrylamide or N-isopropylacrylamide;
   (a3) optionally, a proportion of greater than 0 mol % and less than or equal to 20 mol %, more particularly greater than 0 mol % and less than or equal to 15 mol %, even more particularly greater than or equal to 0 mol % and less than or equal to 10 mol % of monomer units derived from at least one monomer chosen from the elements of the group consisting of (2-hydroxyethyl) acrylate, (2,3-dihydroxypropyl) acrylate, (2-hydroxyethyl) methacrylate, (2,3-dihydroxypropyl) methacrylate and vinylpyrrolidone;
   (a4) optionally, a proportion of greater than 0 mol % and less than or equal to 75 mol % of monomer units derived from at least one monomer chosen from the elements of the group consisting of acrylic acid, methacrylic acid, 2-carboxyethylacrylic acid, itaconic acid, maleic acid and 3-methyl-3-[(1-oxo-2-propenyl)amino]butanoic acid, the carboxylic function of said monomers being in free acid or partially or totally salified form;
   (a5) optionally, a proportion of greater than 0 mol % and of less than or equal to 5 mol % of at least one monomer of formula (M1):

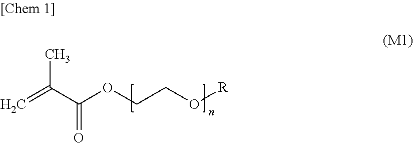

in which R represents a linear or branched alkyl radical including from 8 to 20 carbon atoms and n represents an integer greater than or equal to 0 and less than or equal to 20;
   (a6) a proportion of greater than 0 mol % and of less than or equal to 1 mol % of monomer units resulting from at least one diethylenic or polyethylenic crosslinking monomer (AR); the sum of said molar proportions as monomer units according to a1), a2), a3), a4), a5) and a6) being equal to 100 mol %.

For the purposes of the present invention, the term “salified” means that the acid function present in a monomer is in an anionic form associated in salt form with a cation, notably alkali metal salts, such as the sodium or potassium cations, or such as cations of nitrogenous bases such as the ammonium salt, the lysine salt or the monoethanolamine salt (HOCH₂—CH₂—NH₄⁺). They are preferably sodium or ammonium salts.

The term “at least one diethylenic or polyethylenic crosslinking monomer (AR)” notably denotes, in the definition of said crosslinked anionic polyelectrolyte (AP1), a monomer chosen from the elements of the group consisting of methylenebis(acrylamide), ethylene glycol dimethacrylate, diethylene glycol diacrylate, ethylene glycol diacrylate, diallylurea, triallylamine, trimethylolpropane triacrylate, diallyloxyacetic acid or a salt thereof, such as sodium diallyloxyacetate, or a mixture of these compounds; and more particularly a monomer chosen from ethylene glycol dimethacrylate, triallylamine, trimethylolpropane triacrylate or methylenebis(acrylamide) or a mixture of these compounds.

According to another particular aspect of the present invention, the pharmaceutical composition (E₁) for topical use is characterized in that said crosslinking monomer (AR) as defined above is used in a molar proportion of less than or equal to 0.5%, more particularly less than or equal to 0.25% and most particularly less than or equal to 0.1%; it is more particularly greater than or equal to 0.005 mol %.

The crosslinked anionic polyelectrolyte (AP1) used in the pharmaceutical composition (E₁) for topical use which is a subject of the present invention may also comprise various additives such as complexing agents, transfer agents or chain-limiting agents.

The crosslinked anionic polyelectrolyte (AP1) used in the pharmaceutical composition (E₁) for topical use which is a subject of the present invention may be prepared by performing a radical polymerization process known to those skilled in the art, for instance processes of solution polymerization, suspension polymerization, inverse suspension polymerization, emulsion polymerization, inverse emulsion polymerization or polymerization in solvent medium followed by a step of precipitation of the polymer formed.

According to a more particular aspect, the crosslinked anionic polyelectrolyte (AP1) used in the pharmaceutical composition (E₁) for topical use which is a subject of the present invention may be prepared by performing a process of polymerization in solvent medium followed by a step of precipitation of the polymer formed, or of inverse emulsion polymerization optionally followed by a step of concentration and/or atomization.

According to a more particular aspect, the crosslinked anionic polyelectrolyte (AP1) used in the pharmaceutical composition (E₁) for topical use which is a subject of the present invention may be prepared according to one of the processes described above and may involve the use of transfer agents or chain-limiting agents. The transfer or chain-limiting agents are more particularly chosen from the group consisting of sodium hypophosphite, alcohols of low molecular weight, for example methanol, ethanol, 1-propanol, isopropanol or butanol, thiols, for example 2-mercaptoethanol, transfer agents comprising a sulfate functional group, for example sodium methallylsulfonate, or mixtures of said transfer agents. The transfer or chain-limiting agents are more particularly used in molar proportions, expressed relative to the total number of moles of monomers employed, of from 0.001 mol % to 1 mol %, more particularly from 0.001 mol % to 0.5 mol % and most particularly from 0.001 mol % to 0.1 mol %.

According to another particular aspect of the present invention, said crosslinked anionic polyelectrolyte (AP1) is an element of the group consisting of a homopolymer of 2-methyl-2-[(1-oxo-2-propenyl)amino]-1-propanesulfonic acid partially or totally salified in sodium salt or ammonium salt form, crosslinked by triallylamine and/or methylenebis(acrylamide); a copolymer of 2-methyl-2-[(1-oxo-2-propenyl)amino]-1-propanesulfonic acid partially or totally salified in sodium salt or ammonium salt form and of acrylic acid partially or totally salified in sodium salt or ammonium salt form, crosslinked by triallylamine and/or methylenebis(acrylamide); a copolymer of 2-methyl-2-[(1-oxo-2-propenyl)amino]-1-propanesulfonic acid (γ) partially or totally salified in sodium salt or ammonium salt form and of acrylic acid (δ) partially or totally salified in sodium salt form in a mole ratio (γ)/(δ) of greater than or equal to 30/70 and less than or equal to 90/10, crosslinked by triallylamine and/or methylenebis(acrylamide); a copolymer of 2-methyl-2-[(1-oxo-2-propenyl)amino]-1-propanesulfonic acid (γ) partially or totally salified in sodium salt form and of acrylic acid (δ) partially or totally salified in sodium salt form in a mole ratio (γ)/(δ) of greater than or equal to 40/60 and less than or equal to 90/10, crosslinked by triallylamine and/or methylenebis(acrylamide); a copolymer of 2-methyl-2-[(1-oxo-2-propenyl)amino]-1-propanesulfonic acid (γ) partially or totally salified in sodium salt form and of acrylamide (ε) in a mole ratio (γ)/(ε) of greater than or equal to 30/70 and less than or equal to 90/10, crosslinked by triallylamine and/or methylenebis(acrylamide); a copolymer of 2-methyl-2-[(1-oxo-2-propenyl)amino]-1-propanesulfonic acid (γ) partially or totally salified in sodium salt form and of hydroxyethyl acrylate (ζ) in a mole ratio (γ)/(δ) of greater than or equal to 30/70 and less than or equal to 90/10, crosslinked by triallylamine and/or methylenebis(acrylamide); a terpolymer crosslinked by triallylamine and/or methylenebis(acrylamide) of 2-methyl-2-[(1-oxo-2-propenyl)amino]-1-propanesulfonic acid partially or totally salified in sodium salt or ammonium salt form, of acrylamide and of acrylic acid partially or totally salified in sodium salt or ammonium salt form; a terpolymer crosslinked by triallylamine and/or methylenebis(acrylamide) of 2-methyl-2-[(1-oxo-2-propenyl)amino]-1-propanesulfonic acid partially or totally salified in sodium salt or ammonium salt form in a molar proportion of greater than or equal to 30% and less than or equal to 45%, of acrylamide in a molar proportion of greater than or equal to 45% and less than or equal to 68% and of acrylic acid partially or totally salified in sodium salt or ammonium salt form in a molar proportion of greater than or equal to 2% and less than or equal to 10%; a terpolymer crosslinked by triallylamine and/or methylenebis(acrylamide) of 2-methyl-2-[(1-oxo-2-propenyl)annino]-1-propanesulfonic acid partially or totally salified in sodium salt or ammonium salt form in a molar proportion of greater than or equal to 30% and less than or equal to 45%, of acrylamide in a molar proportion of greater than or equal to 47% and less than or equal to 68% and of acrylic acid partially or totally salified in sodium salt or ammonium salt form in a molar proportion of greater than or equal to 2% and less than or equal to 8%; a terpolymer crosslinked by trimethylolpropane triacrylate and/or triallylamine and/or methylenebis(acrylamide) of 2-methyl-2-[(1-oxo-2-propenyl)amino]-1-propanesulfonic acid partially or totally salified in sodium salt or ammonium salt form in a molar proportion of greater than or equal to 60% and less than or equal to 80%, of N,N-dimethylacrylamide in a molar proportion of greater than or equal to 15% and less than or equal to 39.5% and of tetraethoxylated lauryl methacrylate in a molar proportion of greater than or equal to 0.5% and less than or equal to 5%; a tetrapolymer crosslinked by trimethylolpropane triacrylate and/or triallylamine and/or methylenebis(acrylamide) of 2-methyl-2-[(1-oxo-2-propenyl)amino]-1-propanesulfonic acid partially or totally salified in sodium salt or ammonium salt form in a molar proportion of greater than or equal to 60% and less than or equal to 80%, of N,N-dimethylacrylamide in a molar proportion of greater than or equal to 15% and less than or equal to 39%, of lauryl methacrylate in a molar proportion of greater than or equal to 0.5% and less than or equal to 2.5% and of stearyl methacrylate in a molar proportion of greater than or equal to 0.5% and less than or equal to 2.5%.

According to a particular aspect, the pharmaceutical composition (E1) for topical use as defined previously is characterized in that the anti-inflammatory substance (AI) is chosen from the alkali metal, alkaline-earth metal, ammonium, N,N-dialkylammonium and N,N,N-trialkylammonium salts, for which each of the alkyl groups includes between 1 and 4 carbon atoms, of the elements of the group consisting of 2-[2-(2,6-dichlorophenyl)aminophenyl]ethanoic acid (CAS number=15307-86-5) or diclofenac of formula (AIa):

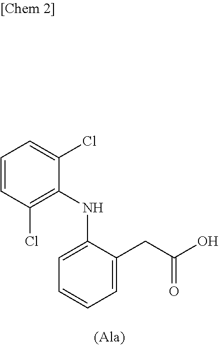 [SMILES: O=C(O)Cc1ccccc1Nc1c(Cl)cccc1Cl]

2-[2-[2-(2,6-dichloroanilino)phenyl]acetyl]oxyacetic acid (CAS number=89796-99-6) or aceclofenac of formula (AIb),

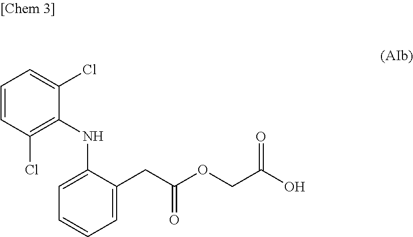 [SMILES: O=C(O)COC(=O)Cc1ccccc1Nc1c(Cl)cccc1Cl]

2-(5-benzoylthiophen-2-yl)propanoic acid (CAS number=33005-95-7 (RS)) or tiaprofenic acid in the R enantiomer form of formula (AIc1) and in the S enantiomer form of formula (AIc2)

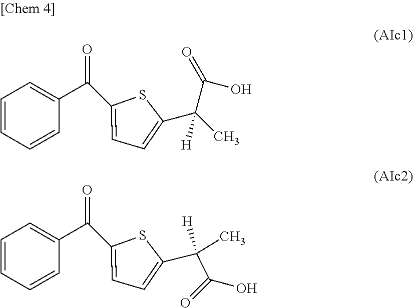 [SMILES: C[C@H](C(=O)O)c1ccc(C(=O)c2ccccc2)s1 ; C[C@@H](C(=O)O)c1ccc(C(=O)c2ccccc2)s1]

2-[4-(2-methylprop-2-enylamino)phenyl]propanoic acid (CAS number=39718-89-3) or alminoprofen of formula (AId),

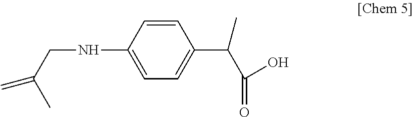 [SMILES: C=C(C)CNc1ccc(C(C)C(=O)O)cc1]

2-(1,8-diethyl-4,9-dihydro-3H-pyrano[3,4-b]indol-1-yl)acetic acid (CAS number=41340-25-4) or etodolac of formula (AIe),

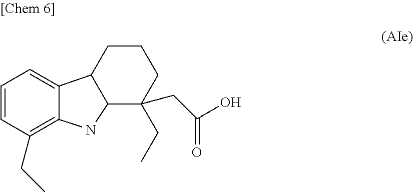 [SMILES: CCc1cccc2c1NC1C2CCCC1(CC)CC(=O)O]

(±)-2-fluoro-α-methyl-(1,1′-biphenyl)-4-acetic acid or flurbiprofen in the R enantiomer form of formula (AIf1) and in the S enantiomer form of formula (AIf2), (CAS number=5104-49-4 (RS)):

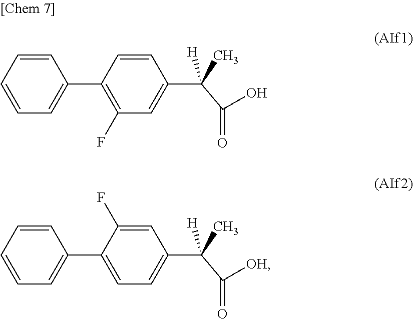 [SMILES: C[C@@H](C(=O)O)c1ccc(-c2ccccc2)c(F)c1 ; C[C@@H](C(=O)O)c1ccc(-c2ccccc2)c(F)c1]

2-[4-(2-methylpropyl)phenyl]propanoic acid or ibuprofen in the R enantiomer form of formula (AIg1) and in the S enantiomer form of formula (AIg2), (CAS number=15687-27-1 (RS)):

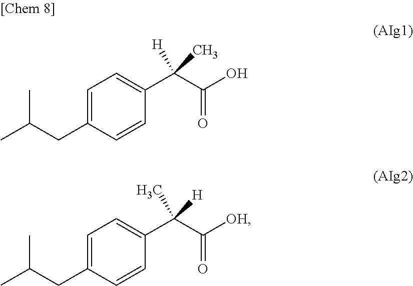

2-(3-benzoylphenyl)propionic acid in the S(+) enantiomer form (CAS number=22161-81-5) and R(−) enantiomer form (CAS number=56105-81-8) and in the form of a racemic mixture (CAS number=22071-15-4) or ketoprofen of formula (AIh),

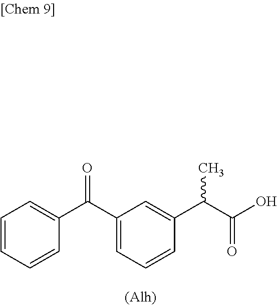 [SMILES: CC(C(=O)O)c1cccc(C(=O)c2ccccc2)c1]

6-methoxy-α-methyl-2-naphthaleneacetic acid or naproxen in the S(+) enantiomer form (CAS number=22204-53-1) of formula (AIi) or in the form of a racemic mixture (CAS number=23981-80-8):

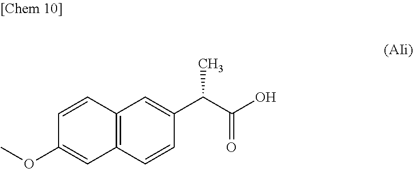 [SMILES: COc1ccc2cc([C@H](C)C(=O)O)ccc2c1]

Preferably, the anti-inflammatory substance is chosen from the sodium salt of 2-[2-(2,6-dichlorophenyl)aminophenyl]ethanoic acid, the diethylammonium salt of 2-[2-(2,6-dichlorophenyl)aminophenyl]ethanoic acid, the sodium salt of 2-[2-[2-(2,6-dichloroanilino)phenyl]acetyl]oxyacetic acid, the diethylammonium salt of 2-[2-[2-(2,6-dichloroanilino)phenyl]acetyl]oxyacetic acid, the sodium salt of 2-[4-(2-methylpropyl)phenyl]propanoic acid, and the sodium salt of 2-(3-benzoylphenyl)propionic acid.

Even more preferentially, the anti-inflammatory substance is the sodium salt of 2-[2-(2,6-dichlorophenyl)aminophenyl]ethanoic acid.

According to a more particular aspect, a subject of the invention is a pharmaceutical composition (E₁) for topical use as defined previously, characterized in that the fatty phase (A2) comprises, per 100% of its mass:
from 1.25% to 25% by mass, more particularly from 1.25% to 20% by mass, of an emulsifying system (S) comprising, per 100% by mass of said emulsifying system (S):
   from 12% to 88% by mass, more particularly from 15% to 85% by mass and even more particularly from 20% to 85% by mass of at least one emulsifying surfactant (S₁) selected from the elements of the group consisting of alkylpolyglycoside compositions, alkylpolyglycoside and fatty alcohol compositions, and
   from 12% to 88% by mass, more particularly from 15% to 85% by mass and even more particularly from 20% to 85% by mass of at least one emulsifying surfactant (S₂) selected from the elements of the group consisting of polyglycerol esters, alkoxylated polyglycerol esters, polyglycol polyhydroxystearates, polyglycerol polyhydroxystearates and alkoxylated polyglycerol polyhydroxystearates;
   from 75% to 98.75% by mass, more particularly from 80% to 98.75% by mass, and even more particularly from 82% to 98.75% by mass, of at least one oil and optionally of at least one wax.

In the definition of the pharmaceutical composition (E1) for topical use which is a subject of the present invention, the term “oil” denotes a compound and/or a mixture of compounds which is water-insoluble and liquid at 25° C., and more particularly:
linear alkanes including from 11 to 19 carbon atoms;
branched alkanes including from 7 to 40 carbon atoms, such as isododecane, isopentadecane, isohexadecane, isoheptadecane, isooctadecane, isononadecane or isoeicosane, or mixtures of some of them such as those mentioned below and identified by their INCI name: C₇₋₈ isoparaffin, C₈₋₉ isoparaffin, C₉₋₁₁ isoparaffin, C₉₋₁₂ isoparaffin, C₉₋₁₃ isoparaffin, C₉₋₁₄ isoparaffin, C₉₋₁₆ isoparaffin, C₁₀₋₁₁ isoparaffin, C₁₀₋₁₂ isoparaffin, C₁₀₋₁₃ isoparaffin, C₁₁₋₁₂ isoparaffin, C₁₁₋₁₃ isoparaffin, C₁₁₋₁₄ isoparaffin, C₁₂₋₁₄ isoparaffin, C₁₂₋₂₀ isoparaffin, C₁₃₋₁₄ isoparaffin, C₁₃₋₁₆ isoparaffin;
cycloalkanes optionally substituted with one or more linear or branched alkyl radicals;
white mineral oils, such as the products sold under the following names: Marcol™ 52, Marcol™ 82, Drakeol™ 6VR, Eolane™ 130, Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes including from 15 to 19 carbon atoms, said alkanes being linear alkanes, branched alkanes and cycloalkanes, and more particularly the mixture (M₁) which comprises, per 100% of its mass, a mass proportion of branched alkanes of greater than or equal to 90% and less than or equal to 100%; a mass proportion of linear alkanes of greater than or equal to 0% and less than or equal to 9%, and more particularly less than 5%, and a mass proportion of cycloalkanes of greater than or equal to 0% and less than or equal to 1%, for example the mixtures sold under the name Emogreen™ L15 or Emogreen™ L19;
the fatty alcohol ethers of formula (II):

Z₁-O-Z₂ (II),

in which Z₁and Z₂, which may be identical or different, represent a linear or branched alkyl radical including from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether;
monoesters of fatty acids and of alcohols of formula (III):

R′₁—(C═O)—O—R′₂ (III),

in which R′₁—(C═O) represents a saturated or unsaturated, linear or branched acyl radical including from 8 to 24 carbon atoms, and R′₂ represents, independently of R′₁, a saturated or unsaturated, linear or branched hydrocarbon-based chain including from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate;
diesters of fatty acids and of glycerol of formula (IV) and of formula (V):

R′₃—(C═O)—O—CH₂—CH(OH)—CH₂—O—(C═O)—R′₄ (IV)

R′₅—(C═O)—O—CH₂—CH[O—(C═O)—R′₆]—CH₂—OH (V),

in which formulae (VI) and (VII) R′₃—(C═O), R′₄—(C═O), R′₅—(C═O) and R′₆—(C═O), which may be identical or different, represent a saturated or unsaturated, linear or branched acyl group including from 8 to 24 carbon atoms;
triesters of fatty acids and of glycerol of formula (VI):

R′₇—(C═O)—O—CH₂—CH[O—(C═O)—R″₈]—CH₂—O—(C═O)—R″₉ (VI),

in which R′₇—(C═O), R′₈—(C═O) and R′₉—(C═O), which may be identical or different, represent a saturated or unsaturated, linear or branched acyl group including from 8 to 24 carbon atoms.
plant oils, such as phytosqualane, sweet almond oil, coconut kernel oil, castor oil, jojoba oil, olive oil, rapeseed oil, groundnut oil, sunflower oil, wheat germ oil, maize germ oil, soybean oil, cottonseed oil, alfalfa oil, poppy seed oil, pumpkin seed oil, evening primrose oil, millet oil, barley oil, rye oil, safflower oil, candlenut oil, passionflower oil, hazelnut oil, palm oil, shea butter, apricot kernel oil, beauty-leaf oil, sisymbrium oil, avocado oil, calendula oil, oils derived from flowers or vegetables;
ethoxylated plant oils.

When the pharmaceutical composition (E₁) for topical use as defined above comprises a wax, said wax is more particularly selected from beeswax, carnauba wax, candelilla wax, ouricoury wax, Japan wax, cork fiber wax, sugar cane wax, paraffin waxes, lignite waxes, microcrystalline waxes, lanolin wax; ozokerite; polyethylene wax; silicone waxes; vegetable waxes; fatty alcohols and fatty acids that are solid at room temperature; glycerides that are solid at room temperature.

According to a particular aspect of the present invention, the pharmaceutical composition (E₁) for topical use as defined previously comprises at least one oil chosen from the elements of the group consisting of castor oil, liquid paraffins, cocoyl caprate/caprylate, isopropyl myristate and capric/caprylic triglyceride.

In the definition of the pharmaceutical composition (E₁) for topical use which is a subject of the present invention, the term “alkylpolyglycoside composition” denotes a composition (C₁) represented by formula (VII):

R₁—O-(G)ₓ-H (VII)

in which x, or the mean degree of polymerization, represents a decimal number between 1.05 and 5, G represents a reducing sugar residue, and R₁ represents a saturated or unsaturated, linear or branched aliphatic hydrocarbon-based radical, optionally substituted with one or more hydroxyl groups, including from 12 to 36 carbon atoms, said composition (C₂) consisting of a mixture of compounds represented by formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅)

in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x.

The term “saturated or unsaturated, linear or branched aliphatic hydrocarbon-based radical including from 12 to 36 carbon atoms, optionally substituted with one or more hydroxyl groups” denotes, for the radical R₁ in formula (VII) as defined above:
saturated linear alkyl radicals, for example n-dodecyl, n-tridecyl, n-tetradecyl, n-pentadecyl, n-hexadecyl, n-heptadecyl, n-octadecyl, n-nonadecyl, n-eicosyl and n-docosyl radicals;
unsaturated linear radicals such as dodecenyl, tridecenyl, tetradecenyl, pentadecenyl, hexadecenyl, heptadecenyl, octadecenyl, nonadecenyl, eicosenyl, docosenyl, 4-dodecenyl and 5-dodecenyl radicals;
saturated or unsaturated, linear or branched aliphatic radicals including from 12 to 36 carbon atoms substituted with one or two hydroxyl groups, such as hydroxydodecyl, hydroxytetradecyl, hydroxyhexadecyl, hydroxyoctadecyl, hydroxyeicosyl and hydroxydocosyl radicals, for example the 12-hydroxyoctadecyl radical;
radicals derived from the isoalkanols of formula (1):

(CH₃)(CH₃)CH—(CH₂)ᵣ—CH₂—OH (1)

in which r represents an integer between 8 and 20, for example the isodecyl, isoundecyl, isododecyl, isotridecyl, isotetradecyl, isopentadecyl, isohexadecyl, isopentadecyl, isooctadecyl, isononadecyl, isoeicosyl or isodocosyl radicals;
branched alkyl radicals, resulting from Guerbet alcohols, of formula (2):

CH(CₛH₂ₛ₊₁)(C₁H₂ₜ₊₁)—CH₂—OH (2)

in which t is an integer between 6 and 18, s is an integer between 4 and 18 and the sum s+t is greater than or equal to 10 and less than or equal to 22, for example the 2-butyloctyl, 2-butyldecyl, 2-hexyloctyl, 2-hexyldecyl, 2-octyldecyl, 2-hexyldodecyl, 2-octyldodecyl, 2-decyltetradecyl, 2-dodecylhexadecyl or 2-tetradecyloctadecyl radicals.

The term “reducing sugar” in the definition of formula (VII) as defined above denotes saccharide derivatives that do not have in their structures any glycoside bonds established between an anomeric carbon and the oxygen of an acetal group as defined in the reference publication: “Biochemistry”, Daniel Voet/Judith G. Voet, page 250, John Wiley & Sons, 1990. The oligomeric structure (G)ₓ may exist in any isomeric form, whether it is optical isomerism, geometrical isomerism or regioisomerism; it may also represent a mixture of isomers. In formula (VII) as defined above, the group R₁—O— is linked to G via the anomeric carbon of the saccharide residue, so as to form an acetal function.

According to a particular aspect in the definition of formula (VII) as defined above, G represents a reducing sugar residue chosen from glucose, dextrose, sucrose, fructose, idose, gulose, galactose, maltose, isomaltose, maltotriose, lactose, cellobiose, mannose, ribose, xylose, arabinose, lyxose, allose, altrose, dextran and tallose; and more particularly G represents a reducing sugar residue chosen from glucose, xylose and arabinose residues.

According to an even more particular aspect, in the definition of formula (II) representing composition (C₁ᵢ) included in the composition (E₁) for topical use which is a subject of the present invention, x, or the mean degree of polymerization, represents a decimal number greater than or equal to 1.05 and less than or equal to 2.5, more particularly greater than or equal to 1.05 and less than or equal to 2.0 and even more particularly greater than or equal to 1.25 and less than or equal to 2.0

According to an even more particular aspect, in the definition of formula (VII) as defined above, R₁ represents the radical chosen from at least one of the elements of the group consisting of n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl and 2-decyltetradecyl radicals; G represents a reducing sugar residue chosen from glucose and xylose residues and x represents a decimal number greater than or equal to 1.05 and less than or equal to 2.5.

According to a more particular aspect, a subject of the invention is a pharmaceutical composition (E₁) for topical use as defined previously, characterized in that the emulsifying surfactant (S₁) consists of at least one alkylpolyglycoside composition (C₁) represented by formula (VII):

R₁—O-(G)x-H (VII)

in which x represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the n-dodecyl, n-tetradecyl, n-hexadecyl, n-octadecyl, n-eicosyl and n-behenyl radicals, said composition (C₁) consisting of a mixture of compounds represented by formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅),

in the respective molar proportions a₁, a₂, a₃, a₄ and a₅ such that:
the sum a₁+a₂+a₃₊a₄+a₅ is equal to 1 and that
the sum a₁+2a₂+3a₃+4a₄+5a₅ is equal to x.

According to an even more particular aspect, the composition (E₁) as defined previously is characterized in that said emulsifying surfactant (S₁) consists of at least one alkylpolyglycoside composition (C₁) represented by formula (VII):

R₁—O-(G)ₓ-H (VII)

in which x, or the mean degree of polymerization, represents a decimal number between 1.05 and 2.5, G represents the glucosyl or α,β-D-glucopyranosyl radical, obtained from the removal of the hemiacetal hydroxyl group from α,β-D-glucopyranose, and R₁ represents:
the n-hexadecyl radical and/or the n-octadecyl radical, or
the n-eicosyl radical and/or the n-behenyl radical, or
the n-tetradecyl radical, or
the n-dodecyl radical and/or the n-tetradecyl radical and/or the n-hexadecyl radical and/or the n-octadecyl radical and/or the n-eicosyl radical and/or the n-behenyl radical.

In the definition of the pharmaceutical composition (E₁) for topical use as defined previously, which is a subject of the present invention, the term “alkylpolyglycoside and fatty alcohol composition” denotes a composition (C₂) comprising, per 100% of its mass:
from 10% to 50% by mass, more particularly from 15% to 40% by mass and even more particularly from 20% to 30% by mass of at least one composition (C₁) represented by formula (VII) as defined previously,
from 90% to 50% by mass, more particularly from 85% to 60% by mass and even more particularly from 80% to 70% by mass of at least one fatty alcohol of formula (VIII):

R′₁—OH (VIII),
in which R′₁, which may be identical to or different from R₁, represents a saturated or unsaturated, linear or branched aliphatic hydrocarbon-based radical, optionally substituted with one or more hydroxyl groups, including from 12 to 36 carbon atoms and preferably from 12 to 22 carbon atoms.

According to a particular aspect, in the definition of formula (VII) representing composition (C₁) included in composition (C₂), R₁ represents the radical chosen from n-dodecyl, n-tetradecyl, n-hexadecyl, n-octadecyl, n-eicosyl and n-docosyl radicals, G represents a glucosyl or α,β-D-glucopyranosyl radical obtained from the removal of the hemiacetal hydroxyl group of α,β-D-glucopyranose, and x represents a decimal number greater than or equal to 1.05 and less than or equal to 2.5.

According to a more particular aspect, in the definition of the fatty alcohol of formula (VIII) as defined above, R′₁ represents a radical chosen from n-dodecyl, n-tetradecyl, n-hexadecyl, n-octadecyl, n-eicosyl and n-docosyl radicals.

According to a more particular aspect, a subject of the invention is a pharmaceutical composition (E₁) for topical use as defined previously, characterized in that said emulsifying surfactant (S₁) consists of at least one composition (C₂) comprising, per 100% of its mass:
from 10% to 50% by mass, more particularly from 15% to 40% by mass and even more particularly from 20% to 30% by mass of at least one alkylpolyglycoside composition (C₁) represented by formula (VII):

R₁—O-(G)x-H (VII),

in which x represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the n-dodecyl, n-tetradecyl, n-hexadecyl, n-octadecyl, n-eicosyl and n-behenyl radicals, said composition consisting of a mixture of compounds represented by formulae (VII₁), (VII₂), (VII₃), (VII₄) and (VII₅):

R1—O-(G)1-H (VII1)

R1—O-(G)2-H (VII2)

R1—O-(G)3-H (VII3)

R1—O-(G)4-H (VII4)

R1—O-(G)5-H (VII5)

in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x; and
from 50% to 90% by mass, more particularly from 60% to 85% by mass and even more particularly from 70% to 80% by mass of at least one fatty alcohol of formula (VIII):

R″₁—OH (VIII),

in which R″₁ represents a radical chosen from the elements of the group consisting of the n-dodecyl, n-tetradecyl, n-hexadecyl, n-octadecyl, n-eicosyl and n-behenyl radicals, where R″₁ may be identical to or different from R₁.

According to a more particular aspect, a subject of the invention is a pharmaceutical composition (E₁) for topical use as defined previously, characterized in that the emulsifying surfactant (S₁) consists of at least one alkylpolyglycoside composition (C′₁) represented by formula (IX):

R₁—O-(G)ₓ-H (IX)

in which x represents a decimal number between 1.05 and 2.5, G represents a xylosyl or α,β-D-xylopyranosyl radical, obtained from the removal of the hemiacetal hydroxyl group from α,β-D-xylopyranose represents a 2-octyldodecyl radical, said composition (C′₁) consisting of a mixture of compounds represented by formulae (IX₁), (IX₂), (IX₃), (IX₄) and (IX₅):

R₁—O-(G)₁-H (IX₁)

R₁—O-(G)₂-H (IX₂)

R₁—O-(G)₃-H (IX₃)

R₁—O-(G)₄-H (IX₄)

R₁—O-(G)₅-H (IX₅)

in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x.

According to a more particular aspect, a subject of the invention is a pharmaceutical composition (E₁) for topical use as defined previously, characterized in that said emulsifying surfactant (S₁) consists of at least one composition (C′₂) comprising, per 100% of its mass:
from 10% to 50% by mass, more particularly from 15% to 40% by mass and even more particularly from 20% to 30% by mass of at least one alkylpolyglycoside composition (C′1) represented by formula (X):

R₁—O-(G)ₓ-H (X)

in which x represents a decimal number between 1.05 and 2.5, G represents a xylosyl or α,β-D-xylopyranosyl radical, obtained from the removal of the hemiacetal hydroxyl group from α,β-D-xylopyranose, and R₁ represents a 2-octyldodecyl radical, said composition consisting of a mixture of compounds represented by formulae (X₁), (X₂), (X₃), (X₄) and (X₅):

R₁—O-(G)₁-H (X₁)

R₁—O-(G)₂-H (X₂)

R₁—O-(G)₃-H (X₃)

R₁—O-(G)₄-H (X₄)

R₁—O-(G)₅-H (X₅)

in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x; and
from 50% to 90% by mass, more particularly from 60% to 85% by mass and even more particularly from 70% to 80% by mass of at least one fatty alcohol of formula (XI):

R″′₁—OH (XI),

in which R″′₁ represents a 2-octyldodecyl radical.

In the definition of the pharmaceutical composition (E₁) for topical use which is a subject of the present invention, the term “polyglycerol ester” denotes a compound of formula (XII):

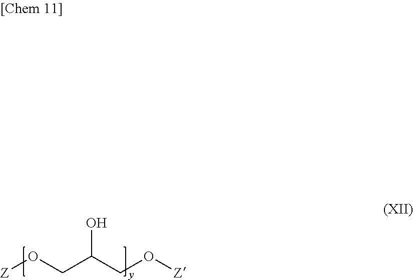

in which Z represents an acyl radical of formula R₂—C(═O)—, in which R₂ represents a saturated or unsaturated, linear or branched aliphatic hydrocarbon-based radical, including from 11 to 35 carbon atoms and more particularly a radical chosen from dodecanoyl, tetradecanoyl, hexadecanoyl, octadecanoyl, eicosanoyl, docosanoyl, oleyl, linoleyl, linolenoyl and isostearyl radicals, Z′ represents the acyl radical of formula R₂—C(═O)— as defined above, with Z′ being identical to or different from Z, or a hydrogen atom, and y represents an integer greater than than or equal to 2 and less than or equal to 20.

According to a more particular aspect, the compound of formula (XII) is chosen from the elements of the group consisting of decaglyceryl oleate, decaglyceryl isostearate, decaglyceryl monolaurate, decaglyceryl monolinoleate and decaglyceryl monomyristate. In the definition of the pharmaceutical composition (E₁) for topical use which is a subject of the present invention, the term “alkoxylated polyglycerol ester” denotes a compound of formula (XIII):

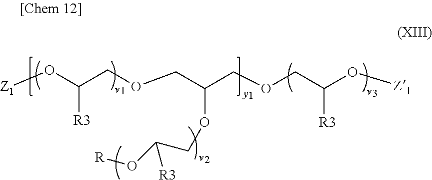

in which Z₁ represents an acyl radical of formula R′₂—C(═O)—, in which R′₂ represents a saturated or unsaturated, linear or branched aliphatic hydrocarbon-based radical, including from 11 to 35 carbon atoms, and more particularly a radical chosen from dodecanoyl, tetradecanoyl, hexadecanoyl, octadecanoyl, eicosanoyl, docosanoyl, oleyl, linoleyl, linolenoyl and isostearyl radicals, represents the acyl radical of formula R′₂—C(═O)— as defined above, with Z₁′ being identical to or different from Z₁, or a hydrogen atom, R₃ represents a hydrogen atom, a methyl radical or an ethyl radical, y₁ represents an integer greater than than or equal to 2 and less than or equal to 20, v₁, v₂ and v₃, which may be identical or different, represent an integer greater than or equal to 0 and less than or equal to 50, and the sum [(y₁. v₁)+(y₁. v₂)+v₃)] is an integer greater than or equal to 1 and less than or equal to 50. In the definition of the pharmaceutical composition (E₁) for topical use which is a subject of the present invention, the term “polyglycol polyhydroxystearate” denotes a compound of formula (XIV):

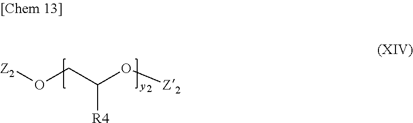

in which y₂ represents an integer greater than or equal to 2 and less than or equal to 50, R₄ represents a hydrogen atom, a methyl radical or an ethyl radical, and Z₂ represents a radical of formula (XV):

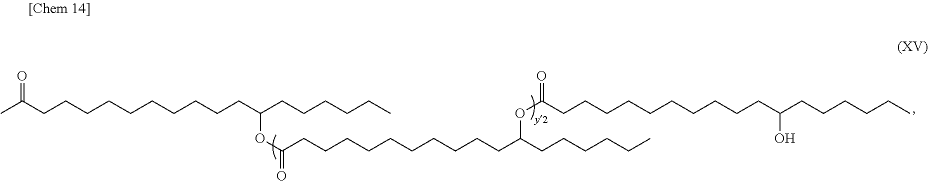

in which y′₂ represents an integer greater than or equal to 0 and less than or equal to 10, more particularly greater than or equal to 1 and less than or equal to 10 and Z′₂ represents a radical of formula (XV) as defined above, with Z₂′ being identical to or different from Z₂, or a hydrogen atom.

In the definition of the pharmaceutical composition (E₁) for topical use which is a subject of the present invention, the term “polyglyceryl polyhydroxystearate” denotes a compound represented by formula (XVI):

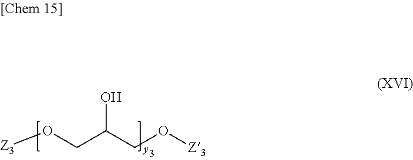

in which Z₃ represents a radical of formula (XVI) as defined above, Z′₃ represents a radical of formula (XV) as defined above, with Z₃′ being identical to or different from Z₃, or a hydrogen atom, y₃ represents an integer greater than or equal to 2 and less than or equal to 20. In the definition of the pharmaceutical composition (E₁) for topical use which is a subject of the present invention, the term “alkoxylated polyglyceryl polyhydroxystearate” denotes a compound represented by formula (XVII):

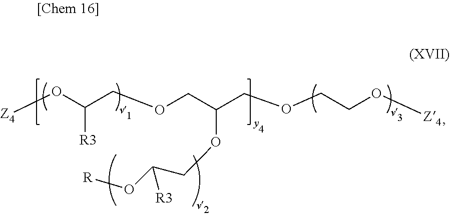

in which Z₄ represents a radical of formula (XV) as defined above, Z′₄ represents a radical of formula (XV) as defined above, with Z₄′ being identical to or different from Z₄, or a hydrogen atom, y₄ represents an integer greater than or equal to 2 and less than or equal to 20, v′₁, v′₂ and v′₃, which may be identical or different, represent an integer greater than or equal to 0 and less than or equal to 50, and the sum [(y₄. v′₁)+(y₄. v′₂)+v′₃)] is an integer greater than or equal to 1 and less than or equal to 50.

According to a more particular aspect, a subject of the invention is a pharmaceutical composition (E₁) for topical use as defined previously, characterized in that the emulsifying surfactant (S₂) consists of at least one polyglycol polyhydroxystearate represented by formula (XIV):

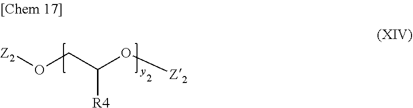

in which y2 represents an integer greater than or equal to 2 and less than or equal to 50, R₄ represents a hydrogen atom, a methyl radical or an ethyl radical, and Z₂ represents a radical of formula (XV):

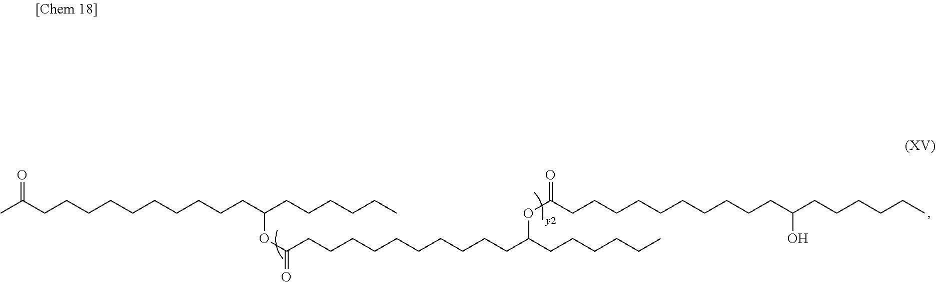

in which y′₂ represents an integer greater than or equal to 0 and less than or equal to 10, more particularly greater than or equal to 1 and less than or equal to 10, and Z′₂ represents a radical of formula (XV) as defined above, with Z′₂ being identical to or different from Z₂, or a hydrogen atom.

According to another more particular aspect, a subject of the invention is a pharmaceutical composition (E₁) for topical use as defined previously, characterized in that said emulsifying system (S) consists of a composition (C₃) comprising, per 100% of its mass:

from 12% to 88% by mass, more particularly from 15% to 85% by mass and even more particularly from 20% to 32% by mass of at least one emulsifying surfactant (S₁) which consists of at least one composition (C₂) comprising, per 100% of its mass:
from 10% to 50% by mass, more particularly from 15% to 40% by mass and even more particularly from 20% to 30% by mass of at least one alkylpolyglycoside composition (C₁) represented by formula (VIII):

R₁—O-(G)ₓ-H (VIII)

in which x, or the mean degree of polymerization, represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the n-octyl, n-decyl, n-dodecyl, n-tetradecyl, n-hexadecyl, n-octadecyl, n-eicosyl and n-behenyl radicals, said composition consisting of a mixture of compounds represented by formulae (VIII₁), (VIII₂), (VIII₃), (VIII₄) and (VIII₅):

R₁—O-(G)₁-H (VIII₁)

R₁—O-(G)₂-H (VIII₂)

R₁—O-(G)₃-H (VIII₃)

R₁—O-(G)₄-H (VIII₄)

R₁—O-(G)₅-H (VIII₅)

in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x; and
from 90% to 50% by mass, more particularly from 85% to 60% by mass and even more particularly from 80% to 70% by mass of at least one fatty alcohol of formula (IX):

R′₁—OH (IX),

in which R′₁ represents a radical chosen from the elements of the group consisting of the n-octyl, n-decyl, n-dodecyl, n-tetradecyl, n-hexadecyl, n-octadecyl, n-eicosyl and n-behenyl radicals, where R′₁ may be identical to or different from R₁;
from 12% to 88% by mass, more particularly from 15% to 85% by mass and even more particularly from 20% to 32% by mas of at least one emulsifying surfactant (S₂) which consists of at least one polyglycol polyhydroxystearate represented by formula (XIV):

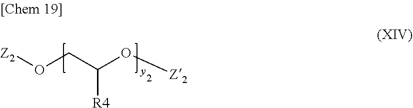

in which y₂ represents an integer greater than or equal to 2 and less than or equal to 50, R₄ represents a hydrogen atom, a methyl radical or an ethyl radical, and Z₂ represents a radical of formula (XV):

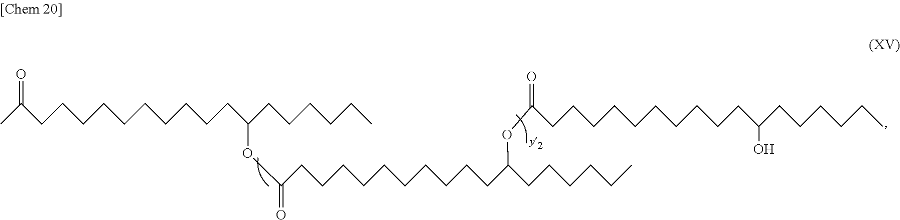

in which y′₂ represents an integer greater than or equal to 0 and less than or equal to 10, more particularly greater than or equal to 1 and less than or equal to 10, and Z′₂ represents a radical of formula (XIII) as defined above, with Z₂′ being identical to or different from Z₂, or a hydrogen atom.

According to another particular aspect, a subject of the invention is a pharmaceutical composition (E₁) for topical use as defined previously, characterized in that the dynamic viscosity of said composition (E₁), measured at a temperature of 20° C. using a Brookfield LVT viscometer at a speed of 6 rpm, is greater than or equal to 500 mPa·s and less than or equal to 40 000 mPa·s.

According to another particular aspect, a subject of the invention is a pharmaceutical composition (E₁) for topical use as defined previously, characterized in that the mass ratio between the emulsifying surfactant (S1) and the emulsifying surfactant (S2) is greater than or equal to ¼ and greater than or equal to 1, preferably greater than or equal to ⅓ and less than or equal to 1, even more preferentially greater than or equal to ⅓ and less than or equal to ½.

A subject of the invention is also a pharmaceutical composition (E1) for topical use according to the invention, for use in human or animal therapy.

According to a particular aspect a subject of the present invention is a pharmaceutical composition (E1) for topical use according to the invention, for reducing and/or eliminating local pains, post-traumatic inflammation of the joints, muscles, tendons or ligaments, localized forms of soft-tissue rheumatism, localized forms of degenerative rheumatic diseases, actinic keratosis caused by overexposure to sunlight, acute migraine, pain associated with bone metastases, fever due to malignant lymphogranulomatosis (Hodgkin's lymphoma), multi-drug resistant E. coli, Shy-Drager syndrome and diabetes mellitus. The pharmaceutical composition (E1) for topical use according to the invention may be packaged in pressurized form in an aerosol device or in a device of “pump-bottle” type, in a tube, in a device equipped with a perforated wall, for example a grille, or in a device equipped with a ball applicator (known as a “roll-on”).

The pharmaceutical composition (E1) for topical use according to the invention may also include excipients and/or active principles commonly used in the field of formulations for topical use, in particular pharmaceutical or dermopharmaceutical formulations.

According to a particular aspect, the pharmaceutical composition (E1) for topical use according to the invention also comprises at least one or more auxiliary compounds chosen from foaming and/or detergent surfactants, thickening and/or gelling surfactants, thickening and/or gelling agents, stabilizers, film-forming compounds, solvents and cosolvents, hydrotropic agents, plasticizers, opacifiers, nacreous agents, superfatting agents, sequestrants, chelating agents, antioxidants, fragrances, essential oils, preserving agents, conditioners, deodorants, mineral fillers or pigments. The composition according to the invention may generally comprise excipients and/or active principles commonly used in the field of formulations for topical, in particular pharmaceutical or dermopharmaceutical, use. Finally, a subject of the invention is a device which is in a form chosen from a jar, a pump-bottle, a wipe, a mask, a transdermal device, a patch, a poultice, a compress, a tube or a spray, said device comprising a pharmaceutical composition (E1) for topical use according to the invention.

As examples of foaming and/or detergent surfactants that may be present in the pharmaceutical composition (E1) for topical use which is a subject of the present invention, mention may be made of topically acceptable anionic, cationic, amphoteric or nonionic foaming and/or detergent surfactants commonly used in this field of activity.

Among the foaming and/or detergent anionic surfactants that may be combined with the pharmaceutical composition (E1) for topical use that is a subject of the present invention, mention may be made of alkali metal salts, alkaline-earth metal salts, ammonium salts, amine salts or amino alcohol salts of alkyl ether sulfates, of alkyl sulfates, of alkylamido ether sulfates, of alkylarylpolyether sulfates, of monoglyceride sulfates, of a-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkyl sulfoacetates, of alkyl sarcosinates, of acylisethionates, of N-acyl taurates, of acyl lactylates, of N-acylamino acid derivatives, of N-acyl peptide derivatives, of N-acyl protein derivatives or of fatty acids.

Among the foaming and/or detergent amphoteric surfactants that may be present in the pharmaceutical composition (E1) for topical use which is a subject of the present invention, mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants that may be present in the pharmaceutical composition (E1) for topical use which is a subject of the present invention, mention may be made particularly of quaternary ammonium derivatives.

Among the foaming and/or detergent nonionic surfactants that may be present in the pharmaceutical composition (E1) for topical use which is a subject of the present invention, mention may be made more particularly of alkylpolyglycosides including a linear or branched, saturated or unsaturated aliphatic radical and including from 8 to 12 carbon atoms; castor oil derivatives, polysorbates, coconut kernel amides and N-alkylamines.

As examples of thickening and/or gelling surfactants that may be present in the pharmaceutical composition (E1) for topical use which is a subject of the present invention, mention may be made of:
optionally alkoxylated fatty esters of alkylpolyglycosides, and most particularly ethoxylated esters of methylpolyglucoside such as PEG 120 methyl glucose trioleate and PEG 120 methyl glucose dioleate sold, respectively, under the names Glucamate™ LT and Glumate™ DOE120;
alkoxylated fatty esters, such as PEG 150 pentaerythrityl tetrastearate, sold under the name Crothix™ DS53, or PEG 55 propylene glycol oleate, sold under the name Antil™ 141;
carbamates of polyalkylene glycols comprising fatty chains, such as PPG 14 laureth isophoryl dicarbamate, sold under the name Elfacos'1211, or PPG 14 palmeth 60 hexyl dicarbamate, sold under the name Elfacos™ GT2125.

As examples of emulsifying surfactants that may be present in the pharmaceutical composition (E1) for topical use which is a subject of the present invention, mention may be made of nonionic surfactants, anionic surfactants and cationic surfactants.

As examples of emulsifying nonionic surfactants optionally present in the pharmaceutical composition (E1) for topical use which is a subject of the present invention, ethoxylated castor oil and ethoxylated hydrogenated castor oil, for example the product sold under the name Simulsol™ 989; compositions comprising glycerol stearate and stearic acid poly(ethoxylated) with between 5 mol and 150 mol of ethylene oxide, for example the composition comprising stearic acid (ethoxylated) with 135 mol of ethylene oxide and glycerol stearate sold under the name Simulsol™ 165; ethoxylated sorbitan esters, for example the products sold under the name Montanox™; ethoxylated mannitan esters; sucrose esters; methyl glucoside esters.

As examples of emulsifying anionic surfactants that may be present in the pharmaceutical composition (E1) for topical use which is a subject of the present invention, mention may be made of decyl phosphate, cetyl phosphate sold under the name Amphisol™, glyceryl stearate citrate; cetearyl sulfate; the arachidyl/behenyl phosphates and arachidyl/behenyl alcohols composition sold under the name Sensanov™ WR; soaps, for example sodium stearate or triethanolammonium stearate, or N-acylated derivatives of amino acids which are salified, for instance stearoyl glutamate.

As examples of emulsifying cationic surfactants that may be present in the pharmaceutical composition (E1) for topical use which is a subject of the present invention, mention may be made of amine oxides, quaternium-82 and the surfactants described in patent application WO 96/00719 and mainly those in which the fatty chain comprises at least 16 carbon atoms.

As examples of opacifiers and/or nacreous agents that may be present in composition (E1) for topical use which is a subject of the present invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate and fatty alcohols including from 12 to 22 carbon atoms.

As examples of texturing agents that may be present in the pharmaceutical composition (E1) for topical use which is a subject of the present invention, mention may be made of N-acylamino acid derivatives, for example lauroyl lysine sold under the name Aminohope™ LL, octenyl starch succinate sold under the name Dryflo™, myristyl polyglucoside sold under the name Montanov 14, cellulose fibers, cotton fibers, chitosan fibers, talc, sericite and mica.

As examples of solvents and cosolvents that may be present in the pharmaceutical composition (E1) for topical use which is a subject of the present invention, mention may be made of water, organic solvents, for example dimethyl sulfoxide (DMSO), ethyl acetate, benzyl alcohol, propylene carbonate, glycerol, diglycerol, glycerol oligomers, PEG-400, ethylene glycol, propylene glycol, butylene glycol, hexylene glycol, diethylene glycol, xylitol, erythritol, sorbitol, water-soluble alcohols such as ethanol, isopropanol or butanol, and mixtures of water and of said organic solvents.

As examples of thickening and/or gelling agents that may be present in the pharmaceutical composition (E1) for topical use which is a subject of the present invention, mention may be made of polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=⅕), locust bean gum (DS=¼), tara gum (DS=⅓), guar gum (DS=½) or fenugreek gum (DS=1).

As examples of thickening and/or gelling agents that may be present in the pharmaceutical composition (E1) for topical use which is a subject of the present invention, mention may be made of polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates and karaya gum exudates, and glucosaminoglycans.

As examples of thickening and/or gelling agents that may be present in the pharmaceutical composition (E1) for topical use which is a subject of the present invention, mention may be made of cellulose, cellulose derivatives such as methyl cellulose, ethyl cellulose, hydroxypropyl cellulose, silicates, starch, hydrophilic starch derivatives, and polyurethanes.

As examples of stabilizers that may be present in the pharmaceutical composition (E1) for topical use which is a subject of the present invention, mention may be made of microcrystalline waxes, and more particularly of ozokerite, and mineral salts such as sodium chloride or magnesium chloride.

As examples of thermal or mineral waters which may be combined with the pharmaceutical composition (E1) for topical use which is a subject of the present invention, mention may be made of thermal or mineral waters having a mineralization of at least 300 mg/l, in particular Avene water, Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Rochefort water, Saint Christau water, Les Fumades water and Tercis-les-Bains water.

The pharmaceutical composition (E1) for topical use which is a subject of the present invention and as defined previously is obtained by performing the preparation process comprising the following steps:

A step a) of preparing the fatty phase (A₂) by mixing all of the elements constituting it in the desired proportions. This mixing step is generally performed at a temperature of greater than or equal to 20° C. and less than or equal to 80° C., more particularly greater than or equal to 20° C. and less than or equal to 70° C., and even more particularly greater than or equal to 20° C. and less than or equal to 460° C.; it is performed with mechanical stirring at a moderate speed of greater than or equal to 50 rpm and less than or equal to 100 rpm;

A step b) of preparing the aqueous phase (A₁) from all of the elements constituting it in the desired proportions. This mixing step is generally performed at a temperature of greater than or equal to 20° C. and less than or equal to 80° C., more particularly of greater than or equal to 20° C. and less than or equal to 60° C. and even more particularly of greater than or equal to 20° C. and less than or equal to 40° C.; it is performed under mechanical stirring at a moderate speed of greater than or equal to 500 rpm and less than or equal to 3000 rpm. In particular, the aqueous phase (A₁) obtained on conclusion of step b) has a dynamic viscosity, measured at 20° C. via a viscometer of Brookfield LV type at a speed of 6 rpm, of greater than or equal to 200 mPa·s and less than or equal to 40 000 mPa·s, more particularly of greater than or equal to 1000 mPa·s and less than or equal to 40 000 mPa·s and even more particularly of greater than or equal to 2000 mPa·s and less than or equal to 40 000 mPa·s;

A step c) during which the fatty phase (A₂) is added to the aqueous phase (A₁) at a temperature of greater than or equal to 20° C. and less than or equal to 80° C., more particularly greater than or equal to 20° C. and less than or equal to 60° C., and even more particularly greater than or equal to 20° C. and less than or equal to 40° C., with mechanical stirring at a moderate speed of greater than or equal to 50 rpm and less than or equal to 400 rpm, so as to obtain the pharmaceutical composition (E1) for topical use.

The examples that follow illustrate the invention without, however, limiting it.

### DESCRIPTION OF THE PREFERRED EMBODIMENTS

### Preparation and Evaluation of Emulsions According to the Invention and Comparative Emulsions

### 1) Preparation of the Emulsions According to the Invention and of the Comparative Emulsions

Three emulsions according to the invention, denoted (F₁) to (F₃), and four comparative emulsions, denoted (F′₁) to (F′₃), the mass proportions of the constituents of which are collated in table 1 below, the mass contents of the polyelectrolytes being indicated as a percentage of polymeric solids, are prepared by performing the process below.

The constituents of the fatty phase are introduced successively into a beaker, mixed and brought to a temperature of 20° C. after an 80° C. heating step; the mixing is performed using a mechanical stirrer equipped with an impeller-type stirring spindle at a speed of 100 rpm. The constituents of the dispersed phase are mixed at room temperature in a beaker using a mechanical stirrer at a speed of 2000 rpm and the thickener is then added gradually. The stirring is maintained for a duration which makes it possible to obtain a phase which is in the form of a homogeneous gel. The fatty phase is added in one go to the gel at room temperature and at a moderate stirring speed (75 to 300 rpm) using a stirrer equipped with an anchor-type spindle. This stirring is then maintained for 10 minutes and no cooling step is necessary.

TABLE 1

| | (F₁) | (F₂) | (F₃) | (F′₁) | (F′₂) | (F′₃) |
|---|---|---|---|---|---|---|
| Continuous fatty phase | | | | | | |
| Lanol ™ 2681⁽¹⁾ | 15% | 15% | 15% | 15% | 15% | 15% |
| Sepineo ™ SE 68⁽²⁾ | 1% | 0% | 0% | 0% | 0% | 0% |
| Sepicide ™ HB⁽³⁾ | 1% | 1% | 1% | 1% | 1% | 1% |
| Simaline ™WO⁽⁴⁾ | 3% | 3% | 3% | 3% | 3% | 3% |
| Montane ™80⁽⁶⁾ | 0% | 0% | 0% | 1% | 1% | 1% |
| Montanov ™202⁽⁸⁾ | 0% | 1% | 0% | 0% | 0% | 0% |
| Fluidanov′20X⁽⁹⁾ | 0% | 0% | 1% | 0% | 0% | 0% |
| Dispersed phase | | | | | | |
| Sepineo ™P600⁽⁵⁾ | 4% | 4% | 4% | 4% | 4% | 4% |
| PEG-400⁷ | 0% | 0% | 0% | 0% | 0% | 50% |
| Glycerol | 0% | 0% | 0% | 0% | 60% | 0% |
| Water | 0% | 0% | 0% | 0% | 15% | 25% |
| Diclofenac sodium | 1% | 1% | 1% | 1% | 1%⁽*⁾ | 1%⁽*⁾ |
| 1,2-Propanediol | 75% | 75% | 75% | 75% | 0% | 0% |

⁽¹⁾Lanol ™ 2681, or cocoyl caprylate/caprate.
⁽²⁾Sepineo ™ SE 68 is a mixture comprising, per 100% of its mass, from 78% to 85% by mass of a mixture of n-hexadecanol and n-octadecanol, and from 15% to 22% by mass of a mixture of n-hexadecyl glucoside with a mean degree of polymerization of 1.20 and n-octadecyl glucoside with a mean degree of polymerization of 1.20, used as emulsifying agent.
⁽³⁾Sepicide ™ HB is a mixture of phenoxyethanol, methylparaben, ethylparaben, butylparaben and n-propylparaben, used as a preserving agent.
⁽⁴⁾Simaline ™WO, or PEG 30 Dipolyhydroxystearate, is an emulsifying surfactant.
⁽⁵⁾Sepineo ™ P600 is a self-invertible inverse latex comprising, per 100% of its mass, between 30% and 40% by mass of a crosslinked copolymer of acrylamide and of sodium acryloyldimethyltaurate, used as a thickener.
⁽⁶⁾Montane ™ 80 is a composition comprising sorbitan monooleate, used as a water-in-oil type emulsifier.
⁽⁷⁾PEG-400 is a polyethylene glycol with a molecular weight of about 400 g.mol⁻¹.
⁽⁸⁾Montanov ™ 202 is a mixture comprising, per 100% of its mass, from 80% to 90% by massof a mixture of arachidyl alcohol and behenyl alcohol, and from 10% to 20% by mass of arachidyl polyglucosides with a mean degree of polymerization of 1.20, used as an emulsifier.
⁽⁹⁾Fluidanov ™ 20X is a mixture comprising, per 100% of its mass, from 70% to 90% by massof 2-octyl-1-dodecanol and from 10% to 30% by mass of 2-octyl-1-dodecanyl polyxyloside.
⁽*⁾minimum mass proportion of water that could be added to the respective comparativeemulsions (F′₂) and (F′₃) without either obtaining gelation preventing stirring of said emulsion, or obtaining an emulsion having a heterogeneous appearance that persists despite the stirring phases of the preparation process, or a form for which the dispersedphase is the fatty phase.

2) Demonstration of the Properties of Emulsions (F₁) to (F₃) According to the Invention and of Comparative Emulsions (F′₁) to (F′₃) 2.1 Characterization of the Appearance and Viscosity of Emulsions (F₁) to (F₃) According to the Invention and of Comparative Emulsions (F′₁) to (F′₃).

The emulsions (F₁) to (F₃) and (F′₁) to (F′₃) obtained according to the process described previously are then stored in an insulated climatic chamber regulated at a temperature of 25° C. for 3 months. On conclusion of this period of three months, the appearance (APP) of each emulsion prepared is observed and the dynamic viscosity (μ) of each emulsion is measured (in mPa·s) by means of a viscometer at 25° C. (Brookfield LVT, speed 6). An aliquot of these same emulsions (F₁) to (F₃) and (F′₁) to (F′₃) obtained according to the process described above are also stored in an insulated climatic chamber regulated at a temperature of 45° C. for three months. On conclusion of this period of three months, the appearance (APP) of each emulsion prepared is observed and the dynamic viscosity (μ) of each emulsion is measured (in mPa·s) by means of a viscometer at 25° C. (Brookfield LVT, speed 6).

2.2 Characterization of the Direction of the Emulsions (F₁) to (F₃) According to the Invention and of the Comparative Emulsions (F′₁) to (F′₃).

The conductivity (σ) of the emulsions (F₁) to (F₃) according to the invention and of the emulsions (F′1) to (F′3) is measured at 25° C., after a period of storage of said emulsions of one day in an insulated climatic chamber regulated at a temperature of 25° C., by means of an LF 196™ brand conductivity meter from the company WTW equipped with a TetraCon™ 96 electrode. If, for a given emulsion, (σ)≤0.5 μS.cm⁻¹, the emulsion is considered to be non-conductive and consequently the external phase is not the phase based on 1,2-propanediol but the oily phase. Indeed, the conductivity, measured at 25° C., of 1,2-propanediol is equal to 4400 μS.cm⁻¹.

If, for a given emulsion, (σ)>0.5 μS.cm⁻¹, the emulsion is considered to be conductive and consequently the external phase is not the oily phase but the phase based on 1,2-propanediol or glycerol or PEG-400.

This same measurement of the conductivity of the emulsions (F₁) to (F₃) according to the invention and of the comparative emulsions (F′₁) to (F′₃) is measured at 25° C. after three months at 25° C., and after three months at 45° C.

2.3 Results Obtained for the Emulsions (F₁) to (F₃) According to the Invention and for the Comparative Emulsions (F′₁) to (F′₃).

The evaluation methods described in sections 2.1 and 2.2 were applied to the emulsions (F₁) to (F₃) according to the invention and to the comparative emulsions (F′₁) to (F′₃). The results obtained are collated in table 2 below.

TABLE 2

| | (F₁) | (F₂) | (F₃) | (F′₁) | (F′₂) | (F′₃) |
|---|---|---|---|---|---|---|
| (APP) at | Homogeneous | Homogeneous | Homogeneous | Homogeneous | Homogeneous | Homogeneous |
| 1 day | liquid | liquid | liquid | liquid | liquid | liquid |
| (visual) | | | | | | |
| (σ) at 1 | ≤0.5 μS.cm⁻¹ | ≤0.5 μS.cm⁻¹ | ≤0.5 μS.cm⁻¹ | >0.5 μS.cm⁻¹ | ≤0.5 μS.cm⁻¹ | ≤0.5 μS.cm⁻¹ |
| day at | m-1 | 1 | -1 | | | |
| 25° C. | | | | | | |
| Dispersed | Glycolic | Glycolic | Glycolic | Fatty phase | Glycolic | Glycolic |
| phase | phase | phase | phase | | phase | phase |
| (APP) at | Homogeneous | Homogeneous | Homogeneous | Homogeneous | Homogeneous | Homogeneous |
| 7 days | liquid | liquid | liquid | liquid | liquid | liquid |
| at 25° C. | | | | | | |
| (σ) at 3 | ≤0.5 μS.cm⁻¹ | ≤0.5 μS.cm⁻¹ | ≤0.5 μS.cm⁻¹ | >0.5 μS.cm⁻¹ | ≤0.5 μS.cm⁻¹ | ≤0.5 μS.cm⁻¹ |
| months | | | | | | |
| at 25° C. | | | | | | |
| (μ) at 3 | 40 000 | 44 800 mPa.s | 51 000 | not | not | not |
| months | mPas | | mPa.s | measured | measured | measured |
| at 25° C. | | | | | | |
| (APP) at | Homogeneous | Homogeneous | Homogeneous | Homogeneous | Homogeneous | Homogeneous |
| 3 months | liquid | liquid | liquid | liquid | liquid | liquid |
| at 45° C. | | | | | | |
| (σ) at 3 | ≤0.5 μS.cm⁻¹ | ≤0.5 μS.cm⁻¹ | ≤0.5 μS.cm⁻¹ | >0.5 μS.cm⁻¹ | ≤0.5 μS.cm⁻¹ | ≤0.5 μS.cm⁻¹ |
| months | | | | | | |
| at 45° C. | | | | | | |

### 2.4 Analysis of the Results

The emulsions (F₁) to (F₃) according to the invention are thus characterized by:
a stability of their form, of the type phase based on 1,2-propanediol dispersed in oil, after storage for 3 months at a temperature of 25° C.; the appearance observed after this storage period is still homogeneous;
a stability of their form, of the type phase based on 1,2-propanediol dispersed in oil, after storage for 3 months at a temperature of 45° C.; the appearance observed after this storage period is still homogeneous;

The comparative emulsion (F′₁) comprising sorbitan monooleate as water-in-oil lipophilic surfactant does not make it possible to obtain an emulsion in which the dispersed phase is the phase based on 1,2-propanediol.

The comparative emulsions (F′₂) and (F′₃), also comprising sorbitan monooleate as lipophilic surfactant, make it possible to obtain an emulsion in which the dispersed phase is the phase based on 1,2-propanediol, but a minimum an amount of water is necessary.

1. A pharmaceutical composition (E1) suitable for topical use comprising a gelled phase (A1) dispersed in a continuous phase (A2), said pharmaceutical composition (E1) comprising:
a gelled phase (A1) free of added water and comprising at least one anti-inflammatory substance (AI) and at least one diol including from 3 to 8 carbon atoms and represented either by formula (Iₐ):
R^{a}₁—C(R^{b}₁)(OH)—C(OH)(R^{c}₁)(R^{d}₁) (Iₐ),
in which each of the radicals R^{a}₁, R^{b}₁, R^{c}₁ and R^{d}₁ represents, independently of each other, a hydrogen atom or a saturated aliphatic radical including from 1 to 5 carbon atoms, or by formula (I_{b}):
R^{a}₁—C(R^{b}₁)(OH)—[C(OH)(R^{e}₁)(R^{f}₁)]ₜ—C(OH)(R^{c}₁)(R^{d}₁) (I_{b}),
in which t is equal to 1, 2 or 3, each of the radicals R^{a}₁, R^{b}₁, R^{c}₁, R^{d}₁, R^{e}₁ and R^{f}1 represents, independently of each other, a hydrogen atom or a saturated aliphatic radical including from 1 to 5 carbon atoms, it being understood that at least one of the radicals R^{a}₁ or R^{b}₁ and/or at least one of the radicals R^{c}₁ or R^{d}₁ do not represent a hydrogen atom;
a fatty phase (A2) comprising at least one oil and an emulsifying system (S) comprising a combination of at least one emulsifying surfactant (S1) and at least one emulsifying surfactant (S2).

2. The pharmaceutical composition (E1) suitable for topical use as claimed in claim 1, comprising, per 100% of its mass:
from 60% to 98% by mass of the gelled phase (A1), and
from 2% to 40% by mass of the fatty phase (A2).

3. The pharmaceutical composition (E1) suitable for topical use as claimed in claim 1, wherein:
the emulsifying surfactant (S1) is selected from the elements of the group consisting of alkylpolyglycoside compositions, and alkylpolyglycoside and fatty alcohol compositions, and
the emulsifying surfactant (S2) is selected from the elements of the group consisting of polyglycerol esters, alkoxylated polyglycerol esters, polyglycol polyhydroxystearates, polyglycerol polyhydroxystearates and alkoxylated polyglycerol polyhydroxystearates.

4. The pharmaceutical composition (E1) suitable for topical use as claimed in claim 1, wherein the gelled phase (A1) comprises, per 100% of its mass:
from 0.625% to 10% by mass of a crosslinked anionic polyelectrolyte (API),
from 0.625% to 5% by mass of at least one anti-inflammatory substance (AI),
from 85% to 98.75% by mass of at least one diol including from 3 to 8 carbon atoms and represented either by formula (Iₐ), or by formula (I_{b}).

5. The pharmaceutical composition (E1) suitable for topical use as claimed in claim 1, wherein said diol including from 3 to 8 carbon atoms and represented either by formula (Ia) or by formula (Ib) is chosen from 1,2-propanediol, 1,2-butanediol, 1,3-butanediol, 1,2-pentanediol, 1,2-hexanediol, 1,2-octanediol, 2,3-butanediol, 2,3-pentanediol, 2,3-hexanediol, 2,5-hexanediol or 2-methyl-2,4-pentanediol.

6. The pharmaceutical composition (E1) suitable for topical use as claimed in claim 5, wherein said diol including from 3 to 8 carbon atoms and represented either by formula (Iₐ) or by formula (I_{b}) is chosen from 1,2-propanediol, 1,2-butanediol, 1,3-butanediol, 1,2-pentanediol, 1,2-hexanediol or 2-methyl-2,4-pentanediol.

7. The pharmaceutical composition (E1) suitable for topical use as claimed in claim 1, wherein the crosslinked anionic polyelectrolyte (AP1) comprises a proportion of greater than or equal to 25 mol % of monomer units derived from 2-methyl-2-[(1-oxo-2-propenyl)amino]-1-propanesulfonic acid in free acid or partially or totally salified form.

8. The pharmaceutical composition (E1) for suitable topical use as claimed in claim 1, wherein the anti-inflammatory substance (AI) is chosen from the alkali metal, alkaline-earth metal, ammonium, N,N-dialkylammonium and N,N,N-trialkylammonium salts for which each of the alkyl groups includes between 1 and 4 carbon atoms, of the elements of the group consisting of 2-[2-(2,6-dichlorophenyl)aminophenyl]ethanoic acid (CAS number=15307-86-5) or diclofenac of formula (AIa): 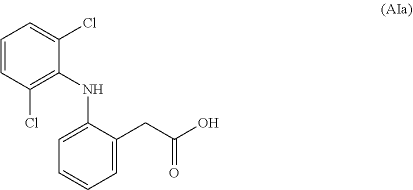 [SMILES: O=C(O)Cc1ccccc1Nc1c(Cl)cccc1Cl]
2-[2-[2-(2,6-dichloroanilino)phenyl]acetyl]oxyacetic acid (CAS number=89796-99-6) or aceclofenac of formula (AIb), 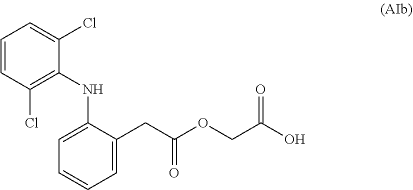 [SMILES: O=C(O)COC(=O)Cc1ccccc1Nc1c(Cl)cccc1Cl]
2-(5-benzoylthiophen-2-yl)propanoic acid (CAS number=33005-95-7 (RS)) or tiaprofenic acid in the R enantiomer form of formula (AIc1) and in the S enantiomer form of formula (AIc2) 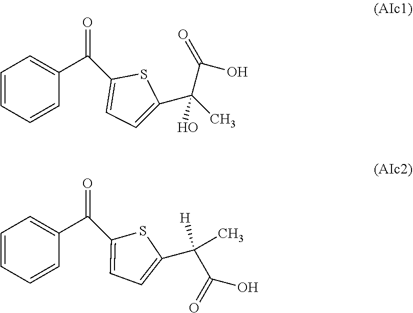 [SMILES: C[C@](O)(C(=O)O)c1ccc(C(=O)c2ccccc2)s1 ; C[C@@H](C(=O)O)c1ccc(C(=O)c2ccccc2)s1]
2-[4-(2-methylprop-2-enylamino)phenyl]propanoic acid (CAS number=39718-89-3) or alminoprofen of formula (AId), 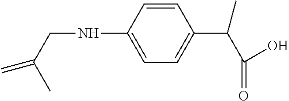 [SMILES: C=C(C)CNc1ccc(C(C)C(=O)O)cc1]
2-(1,8-diethyl-4,9-dihydro-3H-pyrano[3,4-b]indol-1-yl)acetic acid (CAS number=41340-25-4) or etodolac of formula (AIe), 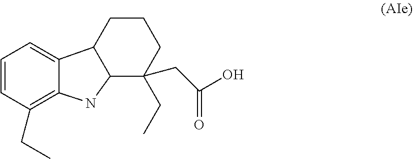 [SMILES: CCc1cccc2c1NC1C2CCCC1(CC)CC(=O)O]
(±)-2-fluoro-α-methyl-(1,1′-biphenyl)-4-acetic acid or flurbiprofen in the R enantiomer form of formula (AIf1) and in the S enantiomer form of formula (AIf2), (CAS number=5104-49-4 (RS)): 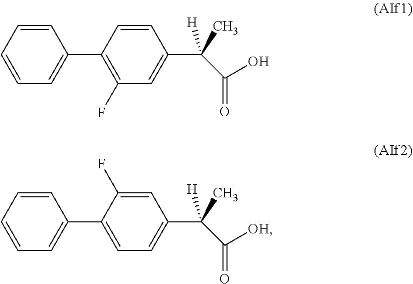 [SMILES: C[C@@H](C(=O)O)c1ccc(-c2ccccc2)c(F)c1 ; C[C@@H](C(=O)O)c1ccc(-c2ccccc2)c(F)c1]
2-[4-(2-methylpropyl)phenyl]propanoic acid or ibuprofen in the R enantiomer form of formula (AIg1) and in the S enantiomer form of formula (AIg2), (CAS number=15687-27-1 (RS)): 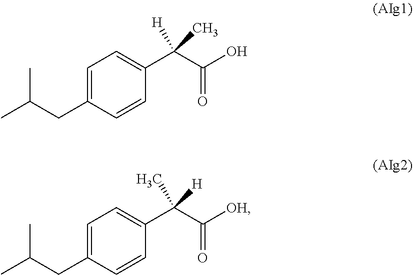
2-(3-benzoylphenyl)propionic acid in the S(+) enantiomer form (CAS number=22161-81-5) and R(−) enantiomer form (CAS number=56105-81-8) and in the form of a racemic mixture (CAS number=22071-15-4) or ketoprofen of formula (AIh), 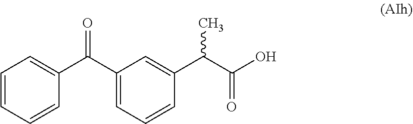 [SMILES: CC(C(=O)O)c1cccc(C(=O)c2ccccc2)c1]
6-methoxy-α-methyl-2-naphthaleneacetic acid or naproxen in the S(+) enantiomer form (CAS number=22204-53-1) of formula (Ii) or in the form of a racemic mixture (CAS number=23981-80-8):

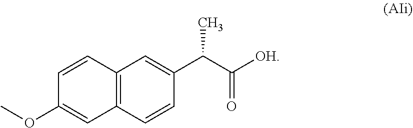 [SMILES: COc1ccc2cc([C@H](C)C(=O)O)ccc2c1]

9. The pharmaceutical composition suitable for topical use as claimed in claim 1, wherein the anti-inflammatory substance (AI) is chosen from the sodium salt of 2-[2-(2,6-dichlorophenyl)aminophenyl]ethanoic acid, the diethylammonium salt of 2-[2-(2,6-dichlorophenyl)aminophenyl]ethanoic acid, the sodium salt of 2-[2-[2-(2,6-dichloroanilino)phenyl]acetyl]oxyacetic acid, the diethylammonium salt of 2-[2-[2-(2,6-dichloroanilino)phenyl]acetyl]oxyacetic acid, the sodium salt of 2-[4-(2-methylpropyl)phenyl]propanoic acid, and the sodium salt of 2-(3-benzoylphenyl)propionic acid.

10. The pharmaceutical composition (E1) suitable for topical use as claimed in claim 1, wherein the fatty phase (A2) comprises, per 100% of its mass:
from 1.25% to 25% by mass of an emulsifying system (S) comprising, per 100% by mass of said emulsifying system (S):
from 12% to 88% by mass of at least one emulsifying surfactant (S₁) selected from the elements of the group consisting of alkylpolyglycoside compositions, alkylpolyglycoside and fatty alcohol compositions, and
from 12% to 88% by mass of at least one emulsifying surfactant (S2) selected from the elements of the group consisting of polyglycerol esters, alkoxylated polyglycerol esters, polyglycol polyhydroxystearates, polyglycerol polyhydroxystearates and alkoxylated polyglycerol polyhydroxystearates;
from 75% to 98.75% by mass of at least one oil.

11. The pharmaceutical composition (E1) suitable for topical use as claimed in claim 1, wherein the emulsifying surfactant (S₁) consists of at least one alkylpolyglycoside composition (C₁) represented by formula (VII):
R₁—O-(G)x-H (VII)
in which x represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the n-dodecyl, n-tetradecyl, n-hexadecyl, n-octadecyl, n-eicosyl and n-behenyl radicals, said composition (C₁) consisting of a mixture of compounds represented by formulae (VII₁), (VII₂), (VII₃), (VII₄) and (VII₅):
R₁—O-(G)₁-H (VII₁)
R₁—O-(G)₂-H (VII₂)
R₁—O-(G)₃-H (VII₃)
R₁—O-(G)₄-H (VII₄)
R₁—O-(G)₅-H (VII₅),
in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x.

12. The pharmaceutical composition (E1) suitable for topical use as claimed in claim 1, wherein the emulsifying surfactant (S₁) consists of at least one composition (C₂) comprising, per 100% of its mass:
from 10% to 50% by mass of at least one alkylpolyglycoside composition (C₁) represented by formula (VII):
R₁—O-(G)ₓ-H (VII),
in which x represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the n-dodecyl, n-tetradecyl, n-hexadecyl, n-octadecyl, n-eicosyl and n-behenyl radicals, said composition consisting of a mixture of compounds represented by formulae (VII₁), (VII₂), (VII₃), (VII₄) and (VII₅):
R₁—O-(G)1-H (VII1)
R₁—O-(G)2-H (VII2)
R₁—O-(G)3-H (VII3)
R₁—O-(G)4-H (VII4)
R₁—O-(G)5-H (VII5)
in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x; and
from 50% to 90% by mass of at least one fatty alcohol of formula (VIII):
R″₁—OH (VIII),
in which R″₁ represents a radical chosen from the elements of the group consisting of the n-dodecyl, n-tetradecyl, n-hexadecyl, n-octadecyl, n-eicosyl and n-behenyl radicals, where R″₁ may be identical to or different from R₁.

13. The pharmaceutical composition (E1) suitable for topical use as defined in claim 1, wherein the emulsifying surfactant (S₁) consists of at least one alkylpolyglycoside composition (C′₁) represented by formula (IX):
R₁—O-(G)ₓ-H (IX)
in which x represents a decimal number between 1.05 and 2.5, G represents a xylosyl or α,β-D-xylopyranosyl radical, obtained from the removal of the hemiacetal hydroxyl group from α,β-D-xylopyranose, and R₁ represents a 2-octyldodecyl radical, said composition (C′₁) consisting of a mixture of compounds represented by formulae (IX₁), (IX₂), (IX₃), (IX₄) and (IX₅):
R₁—O-(G)₁-H (IX₁)
R₂—O-(G)₂-H (IX₂)
R₃—O-(G)₃-H (IX₃)
R₄—O-(G)₄-H (IX₄)
R₅—O-(G)₅-H (IX₅)
in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x.

14. The pharmaceutical composition (E1) suitable for topical use as defined in claim 1, wherein said emulsifying surfactant (S₁) consists of a composition (C′2) comprising, per 100% of its mass:
from 10% to 50% by mass of at least one alkylpolyglycoside composition (C′1) represented by formula (X):
R₁—O-(G)ₓ-H (X)
in which x represents a decimal number between 1.05 and 2.5, G represents a xylosyl or α,β-D-xylopyranosyl radical, obtained from the removal of the hemiacetal hydroxyl group from α,β-D-xylopyranose, and R₁ represents a 2-octyldodecyl radical, said composition consisting of a mixture of compounds represented by formulae (X₁), (X₂), (X₃), (X₄) and (X₅):
R₁—O-(G)₁-H (X₁)
R₁—O-(G)₂-H (X₂)
R₁—O-(G)₃-H (X₃)
R₁—O-(G)₄-H (X₄)
R₁—O-(G)₅-H (X₅)
in the respective molar proportions a₁, a₂, a₃, a₄ and a₅ such that:
the sum a₁+a₂+a₃+a₄+a₅ as is equal to 1 and that
the sum a₁+2a₂+3a₃+4a₄+5a₅ is equal to x; and
from 50% to 90% by mass of at least one fatty alcohol of formula (XI):
R′″₁—OH (XI),
in which R′″₁ represents a 2-octyldodecyl radical.

15. The pharmaceutical composition (E1) suitable for topical use as claimed in claim 1, wherein the emulsifying surfactant (S₂) consists of at least one polyglycol polyhydroxystearate represented by formula (XIV): 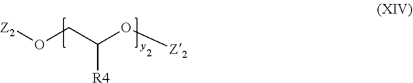
in which y2 represents an integer greater than or equal to 2 and less than or equal to 50, R₄ represents a hydrogen atom, a methyl radical or an ethyl radical, and Z₂ represents a radical of formula (XV): 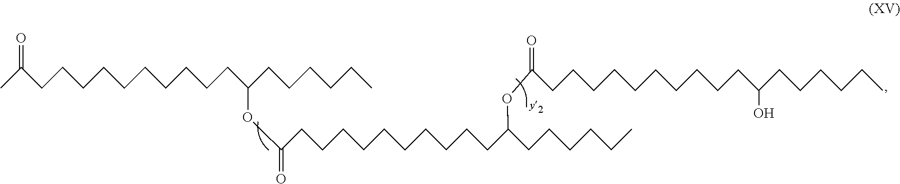
in which y′₂ represents an integer greater than or equal to 0 and less than or equal to 10, and Z′₂ represents a radical of formula (XV) as defined above, with Z′₂ being identical to or different from Z₂, or a hydrogen atom.

16. The pharmaceutical composition (E1) suitable for topical use as claimed in claim 1, wherein the mass ratio between the emulsifying surfactant (S1) and the emulsifying surfactant (S2) is greater than or equal to ¼ and greater than or equal to 1.

17. The pharmaceutical composition (E1) suitable for topical use as claimed in claim 1, for human or animal therapeutic use.

18. A method for reducing and/or eliminating local pains, post-traumatic inflammation of the joints, muscles, tendons or ligaments, localized forms of soft-tissue rheumatism, localized forms of degenerative rheumatic diseases, actinic keratosis caused by overexposure to sunlight, acute migraine, pain associated with bone metastases, fever due to malignant lymphogranulomatosis (Hodgkin's lymphoma), multi-drug resistant E. coli, Shy-Drager syndrome and diabetes mellitus, the method comprising applying an effective dose of the pharmaceutical composition of claim 1 to a patient in need thereof.

19. The pharmaceutical composition (E1) suitable for topical use as claimed in claim 1, further comprising at least one or more auxiliary compounds chosen from foaming and/or detergent surfactants, thickening and/or gelling surfactants, thickening and/or gelling agents, stabilizers, film-forming compounds, solvents and cosolvents, hydrotropic agents, plasticizers, opacifiers, nacreous agents, superfatting agents, sequestrants, chelating agents, antioxidants, fragrances, essential oils, preserving agents, conditioners, deodorants, mineral fillers or pigments.

20. A device which is in a form chosen from a jar, a pump-bottle, a wipe, a mask, a transdermal device, a patch, a poultice, a compress, a tube or a spray, said device comprising a pharmaceutical composition as defined in claim 1.
